(19) United States
(12) Patent Application Publication
(10) Pub. No.: US 2007/0048736 A1
(43) Pub. Date: Mar. 1, 2007

(54) HEPATITIS

(76) Inventors: CHUNGMING CHANG, Miaoli County (TW); KING-SONG JENG, Taipei (TW)

Correspondence Address: FISH & RICHARDSON PC, P.O. BOX 1022, MINNEAPOLIS, MN, 55440-1022, US

(21) Appl. No.: 11/466,831

(22) Filed: Aug. 24, 2006

Related U.S. Application Data
(60) Provisional application No. 60/711,288, filed on Aug. 25, 2005.

(51) Int. Cl. C12Q 1/70; C12Q 1/68; C07H 21/04; C12P 19/34

(52) U.S. Cl. 435/5; 435/6; 536/23.72; 435/325; 435/91.2

(57) ABSTRACT

An isolated nucleic acid containing a mutant hepatitis B virus genome and related methods.

### RELATED APPLICATIONS

This application is a utility from provisional application and claims priority to U.S. Application Ser. No. 60/711,288, filed Aug. 25, 2005, the contents of which are incorporated herein by reference.

### BACKGROUND

Hepatitis can be caused by many infection agents, including hepatitis A, B, C, D, and E viruses and GB virus. Viral hepatitis is the major cause of liver disease. Take chronic hepatitis B (CHB) for example, it is one of the most serious viral infections in humans. It is estimated that more than 350 million people worldwide suffer from CHB and the number is still increasing (Chiaramonte et al., 1999, Cancer 85:2132-7 and Kao et al., 2002, Lancet Infect Dis. 2:395-403). Patients with liver damage resulting from CHB may develop other chronic liver diseases, such as cirrhosis and hepatocellular carcinoma (Mosley et al., 1996 Transfusion 36:776-81.)

In a CHB patient, each infected hepatocyte maintains a pool of covalently closed circular DNA (cccDNA) in the nucleus of a host cell. The cccDNA is implicated in HBV genes expression (Tuttleman et al., 1986, Cell 47:451-60). Pre-genomic RNA (pgRNA) transcribed from the cccDNA, in turn, is reversely transcribed into the relaxed circle (RC) form of the viral DNA within viral particles (Sells et al., J. Virol. 62:2836-44). The mature core particles can either be secreted out of the host cell or re-enter the nucleus to maintain or increase the cccDNA pool (Chisari, 2000, Am. J. Pathol. 156:1117-32; Seeger et al., 2000, Microbiol. Mol. Biol. Rev. 64:51-68; and Wu et al., 1990, Virology 175:255-61). Other than serving as an origin of transcription, HBV cccDNA may also be involved in virus persistence and reappearance in CHB patients after termination of drug treatment (Abdelhamed et al., 2002, J. Virol. 76:8148-60; Lau et al., 2000, Hepatology 32:828-34; and Moraleda et al., 1997, J. Virol. 71:9392-9). Thus, cccDNA plays a pivotal role in the life cycle of HBV. The biosynthesis and biological function of cccDNA are still not entirely clear. There is a need for a suitable model system to study transcriptional regulation of HBV cccDNA and to identify new anti-HBV drugs.

### SUMMARY

This invention relates to a system for monitoring transcription and replication of HBV cccDNA and uses of the system.

One aspect of the invention features an isolated nucleic acid containing a mutant HBV genome that includes a heterologous DNA sequence in the 5′ terminus redundancy region, i.e., the region corresponding to that between direct repeat 1 (DR1) and ε of 5′ end of HBV pregenomic RNA. For example, in HBV strain ayw, the region corresponds to nt 3106-3128 of the genome thereof. One skilled in the art can locate the counterpart regions in other HBV strains based on DNA/RNA sequence homology or RNA secondary structures. The heterologous DNA sequence is 1 to 500 (i.e., any integer number between 1 and 500, inclusive) nucleotides in length. It can be 1-50, 1-20, or 1-10 nucleotides in length. Preferably, the heterologous DNA sequence contains a restriction enzyme site, e.g., a BclI site (5′-TˆG A T C A-3′) . Besides the heterologous DNA sequence, the HBV genome can have other mutations. For example, it can be HBx deficient.

A nucleic acid refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA. An “isolated nucleic acid” is a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to propagate the above-described mutant HBV genome DNA or to generate HBV genomic RNA. For this purpose, one can operatively linked the nucleic acid to suitable regulatory sequences to generate an expression vector.

A vector refers to a nucleic acid molecule capable of transporting another nucleic acid to which it has been linked. The vector can be capable of autonomous replication or integrate into a host DNA. Examples of the vector include a plasmid, cosmid, or viral vector. The vector of this invention includes a nucleic acid in a form suitable for transcription of the nucleic acid in a host cell. Optionally, the vector includes one or more regulatory sequences operatively linked to the nucleic acid sequence to be expressed. A “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of transcription, and the like. The expression vector can be introduced into host cells to produce transcripts of HBV genome.

Also within the scope of this invention is a host cell that contains the above-described nucleic acid. Examples include E. coli cells, insect cells (e.g., using baculovirus expression vectors), yeast cells, or mammalian cells. See e.g., Goeddel, (1990) Gene Expression Technology: Methods in Enzymology 185, Academic Press, San Diego, Calif. To study transcription of HBV cccDNA, the cell should be able to support HBV life cycle. Suitable cells include liver cells or cell lines derived therefrom, e.g., HepG2 or HuH-7 cells. The nucleic acid and host cell can be used to monitor transcription of an HBV cccDNA. To do so, one can (i) obtain a host cell containing the above-described nucleic acid; (ii) culture the host cell to allow for transcription of an HBV cccDNA to generate a population of RNA transcripts, each containing in the 3′ terminus redundancy region an heterologous RNA sequence that is complementary to the heterologous DNA sequence or the complement thereof; and (iii) determine a level of the transcripts. In a preferred embodiment, each of the transcripts contains a second heterologous RNA sequence in the 5′ terminus redundancy region, the second heterologous RNA sequence being identical to the heterologous RNA sequence in the 3′ terminus redundancy region.

One can determine the level of the transcripts by RT-PCR, which generates a DNA product that spans the heterologous RNA sequence. To facilitate detection of the DNA product, the afore-mentioned heterologous DNA sequence can contain a restriction enzyme site, e.g., a BclI site. In that case, the DNA product is subjected to digestion of BclI, and optionally, digestion of SspI.

One can also use the above-described nucleic acid to identify a test compound for treating a chronic infection with HBV. More specifically, one can (1) obtain a host cell containing the nucleic acid; (2) contact the host cell with a test compound in a medium; (3) culture the host cell to allow for transcription of HBV cccDNA to generate a population of RNA transcripts, each containing in the 3′ terminus redundancy region a heterologous RNA sequence that is complementary to the heterologous DNA sequence or the complement thereof; and (4) determine a level of the transcripts. The compound is determined to be effective in treating the infection if the level of the transcripts is lower than that determined in the same manner from a second cell except that the second cell is incubated in a medium free of the compound. Examples of the compound include small molecule compounds, nucleic acids, or peptides (oligopeptides or polypeptides).

The details of one or more embodiments of the invention are set forth in the accompanying drawing and the description below. Other features, objects, and advantages of the invention will be apparent from the description and drawing, and from the claims.

### DESCRIPTION OF DRAWING

FIG. 1 is a diagram showing a plasmid vector containing a mutant HBV genome, which has a BclI site (a heterologous DNA sequence) in the 5′ terminus redundancy region, and use of the vector in monitoring HBV cccDNA transcription.

### DETAILED DESCRIPTION

The present invention relates to HBV, a member of the Hepadnaviridae family virus, which has a circular, partially double-stranded DNA genome and is approximately 3200 nucleotides in length (Ganem et al., 1994, Infect. Agents Dis. 3:85-93 and Seeger et al., 1986, Science 232:477-84). The genome has four defined overlapping open reading frames, which results in transcription and expression of seven different hepatitis B proteins. The genome also contains genetic elements which regulate levels of transcription, determine the polyadenylation site, and mark a transcript for encapsidation into the nucleocapsid.

When infecting a host cell, an HBV virion attaches to and enters the cell. After uncoating, the HBV genome is transported to the nucleus where it is repaired to from the covalently closed circular form (cccDNA). The repair entails completion of the partially double-stranded DNA, removal of the 5′ terminal structures (an RNA primer and the polymerase protein), and covalent ligation of the completed DNA strands.

Once the genome is recircularized, it starts producing various HBV transcripts for HBV protein synthesis and pgRNA. The transcripts can be divided into two categories: subgenomic and genomic. The smaller, subgenomic transcripts serve as mRNA for expressing the X and surface proteins. The larger genomic transcripts, longer than one genome, serve as templates to produce the e, core, and polymerase proteins.

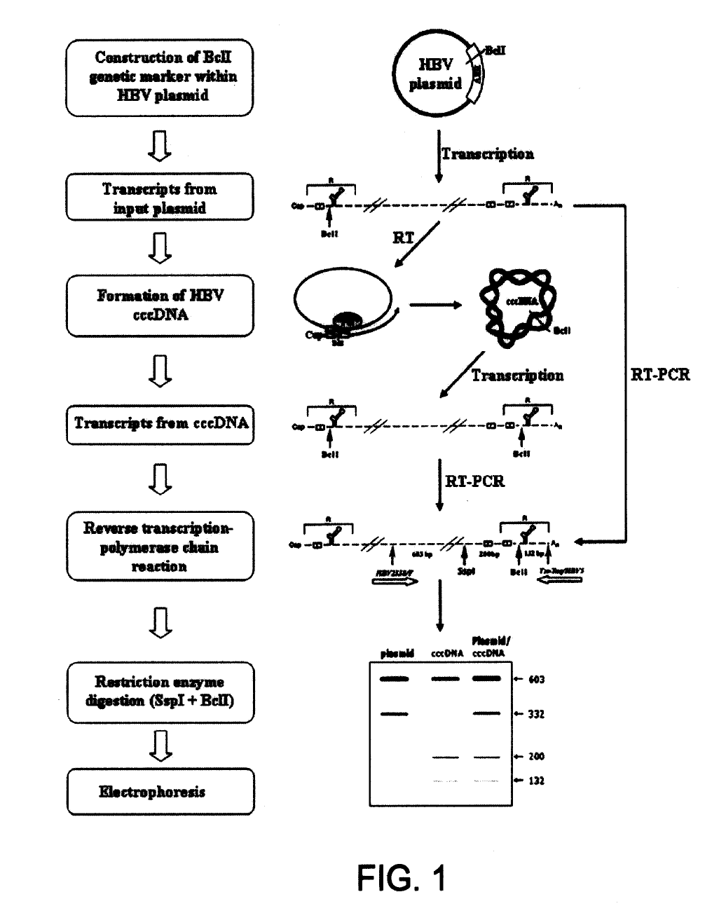

A particular genomic transcript is called pgRNA. It has two terminally-redundant regions. See FIG. 1. Each redundant region (“R”), about 200 nucleotides in length, includes a direct repeat 1 (DR1 or “1”) and a stem-loop. Another copy of DR1 is located near the 3′ end of the pgRNA and denoted as DR2 (“2”). The 3′ stem-loop is bound to by the polymerase, the C-terminus of which interacts with the core protein. Binding of the polymerase protein to the stem-loop results in pgRNA encapsidation.

Also, upon binding of the polymerase to the 3′ stem-loop, the polymerase begins to reverse transcribe the pgRNA template (+) to form (−) DNA strand and is covalently attached onto the growing (−) DNA strand. The polymerase serves as a primer for initiating the reverse transcription. It uses a bulge in the stem-loop as its template for initiating reverse transcription of four bases. These four reverse-transcribed nucleotides share identity with four nucleotides in the 3′ DR1 region. As such, this segment of DNA are transferred to the 3′ DR1 sequence and reverse transcription continues from there. Reverse transcription of the pgRNA generates the (−) DNA strand which is also terminally redundant. As the (−) DNA is synthesized, the copied pgRNA template is degraded by RNase H activity of the polymerase. However, the 15 to 18 capped oligoribonucleotides at the 5′ end of the pgRNA remain intact even after the (−)DNA strand is completed. This cap then serves as an RNA primer for (+)DNA strand synthesis. This RNA primer translocates and base-pairs to the 5′ DR2 region on the (−)DNA strand. Once translocated, synthesis of the (+)DNA strand begins and continues towards the 5′ end of the (−)DNA strand. To complete the (+)DNA strand synthesis, an intramolecular transfer is required to give the (+)DNA strand access to the uncopied portion of the (−)DNA strand. This process is facilitated by the terminal-redundancy found in the (−)DNA strand. Typically, the (+)DNA strand is not completed until re-entry of the virion into another host cell. This yields the characteristic single-stranded gap seen in packaged hepadnaviral DNA.

During the above-described HBV life cycle, a pool of cccDNA is maintained and responsible for virus persistence seen in CHB patients. Understanding of the transcription of cccDNA is therefore expected to shed light on treatments of CHB. Nonetheless, presences of various HBV RNA transcripts in a host cell hinders the study of transcripts specifically derived from cccDNA.

This invention provides a model system to study transcription of HBV cccDNA specifically. It takes the advantage of the duplicated terminal-redundant regions in the cccDNA. Within the scope of this invention is an isolated nucleic acid containing a mutant HBV genome, which contains a heterologous DNA sequence in the 5′ terminus redundancy region. A “heterologous” nucleic acid, gene, or protein is one that originates from a foreign species, or, if from the same species, is substantially modified from its original form. Examples of a nucleic acid sequence heterologous to a HBV genome include sequences generated from (i) point mutating of a naturally occurring HBV genome sequence, (ii) deleting of a segment of naturally occurring HBV genome sequence, (iii) inserting of a non-naturally occurring sequence into a HBV genome sequence, and (iv) a combination thereof. Such a heterologous sequence can include part of a naturally occurring HBV genomic DNA sequence but is not flanked by both of the sequences that flank that part of the DNA sequence in the naturally occurring genome.

To use the nucleic acid to study the transcription of HBV cccDNA, one can transfect it transiently or stably into suitable host cells. Inside the cells, the nucleic acid, like an HBV genome, generates pgRNA, which in turn generates cccDNA in the manner described above. Transcripts directly derived from the nucleic acid contain a copy the heterologous DNA sequence in the 5′ terminus redundancy region. Due to duplication of the terminal-redundant region in cccDNA formation, only cccDNA-derived transcripts contain a copy of the heterologous DNA sequence in the 3′ terminus. Thus, the 3′ heterologous sequence allows one to differentiate transcripts derived from cccDNA and transcripts directly derived from the nucleic acid, as well as other HBV transcripts, e.g., RNA from an integrated HBV genome.

To facilitate the identification, the above-mentioned heterologous DNA sequence can be 1 to 50 nucleic acids in length. In general, the difference between the heterologous sequence and the HBV genomic sequence should be minimized so as to avoid interfering with the HBV life cycle. As shown in the examples below, a single A to G point mutation was introduced into a 1.3-fold HBV genome at nt 3119 to generate a heterologous sequence having a BclI restriction enzyme. Any sequence in the above-mentioned redundant regions can be changed to a heterologous sequence if it does not affect HBV replication and/or RC DNA formation. After transient transfection of p1.3HBcl into host cells, viral pgRNA transcribed from the HBV genome is characterized by the presence of one 5′ BclI restriction site. After double stranded DNA synthesis and cccDNA formation, the BclI site is duplicated. Transcripts from the cccDNA would overlap the BclI site-containing region and therefore contain two BclI sites at the 5′- and 3′-terminus redundant regions, respectively. As a result, the two populations of viral RNAs are identifiable by standard RNA detection techniques, such as amplification-based methods (e.g., RT-PCR), hybridization-based methods, or a combination thereof. To monitor the cccDNA transcription levels, standard quantitative or semi-quantitative RT-PCR methods can be used. Also can used is real-time PCR amplification with aid of a commercially available Real-PCR system (e.g., LightCycler marketed by Roche Molecular Diagnostic.).

The above-described isolated nucleic acid can be used to identify a compound for treating HBV infection. More specifically, one can (i) contact a host cell containing the nucleic acid with a test compound in a medium; (ii) culture the host cell to allow for transcription of HBV cccDNA to generate a population of RNA transcripts, each containing in the 3′ terminus redundancy region a heterologous RNA sequence that is complementary to the heterologous DNA sequence or the complement thereof; and (iii) determine a level of the transcripts. The compound is determined to be effective in treating the infection if the level of the transcript is lower than that determined in the same manner from a second cell except that the second cell is incubated in a medium free of the compound.

Compounds to be screened can be small molecule compounds, nucleic acids, or peptides. They can be obtained using any of the numerous approaches in combinatorial library methods known in the art. Such libraries include: peptide libraries, peptoid libraries (libraries of molecules having the functionalities of peptides, but with a novel, non-peptide backbone that is resistant to enzymatic degradation), spatially addressable parallel solid phase or solution phase libraries, synthetic libraries obtained by deconvolution or affinity chromatography selection, the “one-bead one-compound” libraries, and antibody libraries. See, e.g., Zuckermann et al. (1994) J. Med. Chem. 37, 2678-85; Lam (1997) Anticancer Drug Des. 12, 145; Lam et al. (1991) Nature 354, 82; Houghten et al. (1991) Nature 354, 84; and Songyang et al. (1993) Cell 72, 767. Examples of methods for the synthesis of molecular libraries can be found in the art, for example, in: DeWitt et al. (1993) Proc. Natl. Acad. Sci. USA 90, 6909; Erb et al. (1994) Proc. Natl. Acad. Sci. USA 91, 11422; Zuckermann et al. (1994) J. Med. Chem. 37, 2678; Cho et al. (1993) Science 261, 1303; Carrell et al. (1994) Angew. Chem. Int. Ed. Engl. 33, 2059; Carell et al. (1994) Angew. Chem. Int. Ed. Engl. 33, 2061; and Gallop et al. (1994) J. Med. Chem. 37, 1233. Libraries of compounds may be presented in solution (e.g., Houghten (1992) Biotechniques 13, 412-421), or on beads (Lam (1991) Nature 354, 82-84), chips (Fodor (1993) Nature 364, 555-556), bacteria (U.S. Pat. No. 5,223,409), spores (U.S. Pat. No. 5,223,409), plasmids (Cull et al. (1992) Proc. Natl. Acad. Sci. USA 89, 1865-1869), or phages (Scott and Smith (1990) Science 249, 386-390; Devlin (1990) Science 249, 404-406; Cwirla et al. (1990) Proc. Natl. Acad. Sci. USA 87, 6378-6382; Felici (1991) J. Mol. Biol. 222, 301-310; and U.S. Pat. No. 5,223,409). Compounds to be screened can be known drugs that are used to treat other diseases. Screening known drugs is advantageous, since the toxicity, pharmacokinetics, and side effects data of the drugs are available and all ethical issues have already been solved. The only remaining issue is whether the drugs are effective in treating hepatitis.

A particular compound of interest is a double-stranded ribonucleic acid (dsRNA). This dsRNA can be used to inhibit expression of a protein encoded by the HBV genome, e.g., HBx. The term “dsRNA” refers to a double-stranded ribonucleic acid that silences gene expression via degradation of a targeted RNA sequence, a process known as RNA interference (RNAi). RNAi has been used to silence gene expression in a wide variety of animal models (including C. elegans, zebrafish, and mouse embryos) and in other biological systems (including explanted chick neural cells and mammalian cell culture). See WO99/32619, WO00/44914, WO00/44914, WO00/44895, WO00/63364, and WO01/36646 A1. A dsRNA of this invention can be synthesized by techniques well known in the art. See, e.g., Caruthers et al., 1992, Methods in Enzymology 211, 3-19, Wincott et al., 1995, Nucleic Acids Res. 23, 2677-2684, Wincott et al., 1997, Methods Mol. Bio. 74, 59, Brennan et al., 1998, Biotechnol Bioeng., 61, 33-45, and Brennan, U.S. Pat. No. 6,001,311. The RNA can also be transcribed from an expression vector and isolated using standard techniques. A dsRNA or vector of this invention can be delivered to target cells using method also well known in the art. See, e.g., Akhtar et al., 1992, Trends Cell Bio. 2, 139. For example, it can be introduced into cells using liposomes, hydrogels, cyclodextrins, biodegradable nanocapsules, or bioadhesive microspheres. Alternatively, the RNA or vector is locally delivered by direct injection or by use of an infusion pump. Other approaches include use of various transport and carrier systems, e.g., using conjugates and biodegradable polymers.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

Current cell models for studying HBV replication cannot discriminate between mRNA derived from cccDNA and mRNA derived from transfected plasmid and/or an integrated genome. To identify whether viral transcript from cccDNA, a BclI restriction enzyme site was introduced into the 5′-terminus of the redundancy region of a 1.3-fold HBV genome to generate the plasmid p1.3HBcl.

More specifically, plasmid p1.3HBcl was constructed by a single nucleotide substitution (nt3119; A to G) within the redundancy region of a 1.3 fold-HBV genome (pHBV1.3; ayw subtype; Galibert et al., 1979. Nature 281:646-50. In this plasmid, the transcription of pregenomic RNA (pgRNA) was controlled by the virus's own core promoter and enhancer I and II regulatory elements. The numbers indicate the nucleotide (nt) number from the sequence of full length HBV genome and the start codon of HBc is nt 1.

Plasmid p1.3SspI was constructed by a 9-base (ttaatattt) in-frame insertion at nt3129 of the HBV sequence on the p1.3HBcl backbone vector. Plasmid p1.3HBcl-P2 generated by mutagenesis from p1.3HBcl (nt2024; G to C), which generates a single amino acid substitution (aa540; D to H) within the YMDD motif of the viral polymerase to abolish its reverse transcription ability (Radziwill et al., J. Virol. 64:613-20). Plasmid p1.3HBclI-X⁻ was constructed by an AUG mutation and stop codon (TAATAG) insertion within the ORF of HBx. The HBx mutant plasmid pX-Δ (100-115) has an internal deletion in the region corresponded to amino acid 100-115 residues of pX (Arii et al., 1992, Oncogene 7:397-403; and Takada et al., 1994, Virology 205:503-10.). The BclI site, as a genetic marker, was designed to trace the transcriptional origin of the viral RNA. The p1.3HBcl plasmid was transiently transfected into HepG2 cells. The cells were maintained in Iscove's modified Dulbecco's modified Eagle's medium (DMEM) (Gibco-BRL) containing 10% fetal calf serum (Gibco-BRL), 2 mM L-glutamine, 1% non-essential amino acids, 100 IU penicillin, 100 μg/ml streptomycin, and 2.5 μg/ml fungizone at 37° C. in a 5% CO₂ incubator.

HBV pre-genomic transcripts (pgRNA) transcribed from the plasmid were identified by the fact that each of them contained a BclI restriction site at its 5′-terminus. In contrast, after double stranded DNA synthesis and cccDNA formation, the BclI restriction site was retained within the region between DR1 and epsilon. Transcripts from cccDNA would overlap this region to form the 5′- and 3′-terminus redundancies and both contained the BclI sites. Consequently, the two populations of viral RNAs were identifiable.

cDNA were obtained by reverse transcription of total mRNA with oligo dT 18 mers by SuperScript II reverse transcriptase (Invitrogen). A pair of PCR primers, HBV2338/F′ (agcgtggttatcctgcgttgatg) and T20-Tag/HBV5 (T₂₀-Taq/HBV5 (gcggccgccctgcagtttttttttttttttttttagctc) were designed to specifically amplify the 3′-terminus of HBV transcripts. To exclude the possibility that jumping PCR might generate PCR fragments containing a BclI site derived from the 5′ end of the 3.5-kb HBV RNA carrying BclI sequence, the reverse primer was designed to contain an extremely short HBV sequence with only 5 bases in order to prevent jumping PCR.

RT-PCR amplification generated products of 935 bp in length. As this product contained a single SspI site, it was subjected to double digestion of SspI and BclI to distinguish products amplified from cccDNA-derived RNA and. RNAs transcribed from integrated/plasmid HBV DNA. More specifically, the PCR products were eluted from an agarose gel after electrophoretic separation and restricted by BclI/SspI double digestion. The restricted samples were separated again by electrophoresis on a 1.5% agarose gel in which a standard amount of 603 bp fragment had been added as a loading control. The SspI digestion generated two fragments with sizes of 603 bp and 332 bp. For the products amplified from cccDNA-derived RNA, the 332 bp fragment contained the BclI site and therefore was further cut into two fragments of 200 bp and 132 bp. Thus, the amount of 200 bp or 132 bp fragments were proportional to the amount of mRNA derived from the cccDNA.

To exclude the possibility of mismatch amplification of transfected plasmids, a the above assay was repeated using p1.3HBclI as the PCR template. No PCR products were detectable, indicating that contamination of plasmids would not lead to false positive result.

The above assay was repeated with the p1.3SspI plasimd, a plasmid containing wild type HBV genome, and two control plasmids, p1.3SspI and p1.3HBcl-P2 to verify the above results. The plasmids p1.3SspI and p1.3HBcl-P2 were constructed based on the p1.3HBcl backbone vector. Both of them lost the ability to generate cccDNA molecular.

Plasmid p1.3SspI was identical p1.3HBcl except that a 9-base sequence containing a SspI site was inserted at nt3129 of the HBV genome. After transient transfection of those plasmids into HepG2 cells, Southern blot analysis was carried out to examine the HBV replicative intermediates. It was found that viral replication by p1.3HBcl was almost as good as wild type pHBV1.3, whereas p1.3SspI-transfectants displayed primarily the duplex-linear form DNA (DL) and large amounts of single stranded molecules. In other words, p1.3SspI-transfected HepG2 cells could not produce cccDNA due to absence of its precursor, the relax-circular form DNA (RC). Thus, the RNA prepared from p1.3SspI-transfected HepG2 cells would be an appropriate cccDNA-free control for the RT-PCR and BclI digestion analyses. Plasmid p1.3HBcl-P2 was derived from p1.3HBcl by a single amino acid substitution in the YMDD motif. This YMHD mutant had a significant defect in the reverse transcription ability of the HBV polymerase (Radziwill et al., 1990, J. Virol. 64:613-20.), which completely blocked viral replication and cccDNA production.

To verify the transcriptional ability of cccDNA, HepG2 cells were transfected with pHBV1.3, p1.3HBcl, p1.3HBcl-P2, and p1.3SspI respectively. Three days later, total RNAs were extracted from the cells and subjected to Northern blot analysis. More specifically, the cells were harvested and total RNA was extracted using a Trizol RNA extraction kit (Invitrogen). Fifteen micrograms of total RNA was separated on 1.2% formaldehyde agarose gels by electrophoresis and transferred to nylon membranes. After UV-crossing, the membranes were prehybridized at 42° C. for 4 hours in a pre-hybridization solution (5×SSPE, 0.5% SDS, 10×Denharts' solution, 400 μg/ml salmon sperm DNA, and 100 μg/ml tRNA in 50% formamide) and then hybridized in a hybridization solution (3.5×SSPE, 0.5% SDS, 10×Denharts' solution, 8% dextran sulfate, 300 μg/ml salmon sperm DNA, and 100 μg/ml tRNA in 50% formamide) with P³²-radiolabeled DNA probe (2×10⁸ cpm/μl specific activities, prepared using random oligonucleotide-priming of the whole HBV genome or the glyceraldehyde-3-phosphate dehydrogenase gene). After 16 hours of hybridization, the membranes were washed with 0.2×SSC and 0.1% SDS at 52° C. three times (20 minutes each time), and exposed to X-ray film for 16 hours at −80° C. It was found that the expression profiles of the HBV mRNAs were similar in each group of transfected cells.

The above-described RT-PCR-SspI/BclI double digestion assay was carried out. The 200 bp and 132 bp fragments were obtained from the p1.3HBcl-transfected cells, indicating that some of the partial viral mRNAs found in p1.3HBcl-transfectants were specifically transcribed from cccDNA. When using cDNA prepared from the p1.3HBcl-P2 and p1.3SspI transfectants, the 332 bp fragment could not be cut by BclI, indicating lack of the 3′-terminus BclI marker. The results suggested that the transfectants which did not generate the cccDNA molecule were not able to transfer the 5′-terminus BclI marker to the 3′-terminus redundant region and that the RT-PCR-SspI/BclI double digestion assay can be used to specifically detect transcripts transcribed from cccDNA.

### EXAMPLE 2

Example 1 above described a transient transfection-based assay. In this assay, input plasmids might interfere with studying cccDNA. To solve this problem, several HBV-producing cell lines were generated by stably transfecting HepG2 cells with plasmids carrying the BclI genetic markers. More specifically, HepG2 cells were stably transfected with plasmids containing the 1.3 fold-HBV genome which contained the BclI genetic marker at either the 5′- or the 3′-terminus. The cells were then selected with 1 mg/ml G418.

Based on the position of BclI within integrated viral genome, three types of stable transfectants were obtained. First, stable lines 1.3.ES3, 1.3.ES6, and 1.3.ES8 were characterized by the inclusion of the BclI marker in 5′-terminus redundancy region of integrated viral genome. Second, the 1.3.ES2 cell line brought the BclI site to the 3′-terminus redundancy. Third, a control cell line, 1.3.ES11, was established in which the BclI restriction sites were introduced into both the 5′-terminus and the 3′-terminus redundancy regions of the viral genome.

Preliminary characterization of the chromosomal integration of HBV genome revealed that a single integrated copy of HBV genome was detected in the 1.3.ES2 or 1.3.ES11, since a single band was observed after genomic digestion with HindIII, which was not a restriction site within the HBV genome.

To examine the integrity of the integrated HBV genome, chromosomal DNA was isolated, digested by EcoRI, BamHI, Alw44I, SspI, and AviII (all were able to cut the HBV genome), and subjected to Southern blot using a full-length HBV probe. More specifically, cells were washed twice with an ice-cold GKNP buffer, resuspended in 3 ml of a TEN buffer (10 mM Tris-HCl, pH 7.5, 1 mM EDTA, 100 mM NaCl) containing 0.5% SDS, and incubated at room temperature for 10 minutes. After complete cell lysis, the lysate was treated with 20 μg/ml RNase A at room temperature for 30 minutes. The sample was then treated with 50 mg/ml proteinase K at 52° C. overnight. The supernatant was then extracted with phenol twice, phenol/chloroform once, and chloroform once. Finally, the total DNA (including viral replicative intermediates and cellular genomic DNA) remaining in the supernatant was precipitated with ethanol and dissolved in 1×TE (0.1 mM EDTA, 1 mM Tris-HCl, pH 8.0). The loaded nucleic acid was normalized to equal amounts according to the O.D. value of the DNA. Total DNA or cccDNA was quantified and then separated on 1.2% agarose gels by electrophoresis. The DNAs were denatured twice in a denaturing buffer (0.5 M NaOH, 1.5 M NaCl) and neutralized twice in a neutralizing buffer (10 M Tris-HCl, pH 8.0, 1.5 M NaCl). The DNAs were then transferred to nylon membranes (Hybond-XL, Amersham Pharmacia Biotech) and UV cross-linked. Membranes were prehybridized at 42° C. for 4 hours in a prehybridization solution and then hybridized in a hybridization solution with P³²-radiolabeled DNA probe (2×10⁸ cpm/μl specific activity prepared using random oligonucleotide-priming of the whole HBV genome). After 16 hours of hybridization, the membranes were washed with 0.2×SSC and 0.1% SDS at 52° C. three times (20 minutes each time), and exposed to a X-ray film for 16 hours at −80° C.

If the integrated HBV genome were intact, the restriction fragments should exhibit predicted sizes after hybridizing with. Indeed, the expected bands were observed. For the 1.3.ES2 line, two fragments of 3.3 kb and 2.1 kb were generated after HindIII and EcoRI digestion. However, the 3.3 kb restriction fragment was not found in the 1.3.ES11 after digesting with EcoRI or HindIII/EcoRI. Instead, a 3.1 kb was detected after HindIII/EcoRI double digestion. Because the integrated HBV genome is likely to be intact after restriction enzyme digestion, the results suggest that the proximal sequence located in the down stream region HBV in 1.3.ES11 cells was missing after integration. To examine whether both the 5′ and 3′ end of the integrated HBV genome contained the BclI sites, the DNA was digested with BclI and then probed with an HBV sequence. A unit length 3.2 kb fragment was excised from 1.3.ES11 chromosome DNA, suggesting that BclI sites had been acquired at both the 5′ and 3′ end of the HBV genome during the establishment of the clone. The integrated HBV genome in 1.3.ES8 cells was also likely to be intact and contain two integration sites on the chromosome. Total DNA extracted from the stable lines and Southern blot analysis revealed that those HBV-producing lines with embedded BclI marker could support replicative intermediate formation. Analysis of the viral RNA expression pattern by Northern blot showed the existence of the 3.5 kb pgRNA and 2.1-2.4 kb HBs transcripts that hybridized with the HBV-specific probe. This result suggested that the transcription ability of these HBV stable transfectants was as good as wild type HBV genome.

To demonstrate whether the above-described HBV-producing cells could synthesize cccDNA in their nuclei, DNA was prepared from HBV-stably transfectants according to a method modified from that described in procedure with modification (Yeh et al., 1998, J. Med. Virol. 55:42-50). The cells were washed twice with an ice-cold GKNP washing buffer. The residual washing buffer was removed as completely as possible. The cells were lysed in 3 ml of an Hirt solution (0.6% SDS, 10 mM EDTA, 10 mM Tris-HCl, pH 7.5) for 5 minutes. After completely cell lysis, the cell lysate was mixed with 750 μl of 5N NaCl. After incubating the whole mixture on ice overnight, the mixture was centrifuged at 4000 rpm for 15 minutes at 4° C. The pellet was removed. cccDNA, which was retained in the supernatant, was extracted with phenol twice and phenol/chloroform once and then precipitated by adding two volume of absolute ethanol. The DNA was subjected to Southern analysis.

A control plasmid pUC3.2-HBV (3182 bp0 was constructed and served as a reference for the cccDNA (supercoiled plasmid) and the RC form DNA (nicked plasmid). It was found that the pUC3.2-HBV plasmids and DNA prepared from Hirt supernatant of 1.3.ES8 cells could be separated on the agarose gel and detected by an HBV-specific probe. The upper band migrating at 4.7 kb represents the RC form of the DNA, while the lower one migrating at 2.0 kb is the supercoiled form or covalently closed circular DNA (cccDNA). Both the RC and cccDNA molecules shifted to the 3.2 kb position of duplex linear DNA (DL) upon digesting with the single cutting site enzymes XhoI or SphI, indicating that both RC and cccDNA were of the same length but had different conformations. Upon heating the sample to 85° C. for 5 minutes in a 0.1×TE buffer, the majority of the RC form DNA was shifted down to the position of single-stranded DNA, while the cccDNA was not affected by this treatment. To confirm that the 2.0 kb species DNA from the Hirt extraction was indeed cccDNA, the 85° C.-denatured DNA sample was digested with XhoI or EcoRI (XhoI, SphI, and EcoRI were the single cutting site enzymes within the HBV genome) and subjected to electrophoresis. It was found that the band predicted to be cccDNA was shifted to the position of the linear form DNA. These alterations in gel mobility pointed to the supercoiled nature of the 2.0 kb species DNA, suggesting that the HBV-producing lines were able to produce cccDNA. Judging by the signal intensity comparing to input plasmid, the intensity of the cccDNA was divided by the cell number. This revealed that there were between 4 and 6 copies of the cccDNA within each hepatocyte in the stable lines. To verify the transcription ability of cccDNA in those HBV-producing lines, RT-PCR/BclI assay was performed to measure the transcription ability. In the case of 1.3.ES8, the RT-PCR product was digested with SspI and BclI and generated fragments of 603 bp, 332 bp, 200 bp, and 132 bp. The 332 bp product that lacks the BclI site represented the RNA transcribed from the integrated viral genome, whereas the presence of the two fragments of 200 bp and 132 bp measures those transcripts from the cccDNA. Significantly, the relative intensity of the two fragments derived from the cccDNA RNAs was only 10-20% that from the integrated genome. A similar conclusion was drawn from the other HBV producing cell line, 1.3.ES2. Since the BclI restriction site was located within the 3′-terminus redundant region of viral genome, transcripts from integrated viral genome would contain BclI genetic marker and the RT-PCR product could be further divided into two fragments of 200 bp and 132 bp after restriction. The presence of 332 bp fragment after restriction enzyme digestion indicated the transcripts from cccDNA.

Taking together, the results indicated that transcripts derived from the cccDNA were less than those from the integrated genome. This is highly significant as there are between 4 and 6 copies of the cccDNA present in a single cell and only between 1 and 2 integrated HBV copies. Thus, the transcription ability of the cccDNA was much poorer than that of the integrated HBV genome.

Comparing the relative intensity of cccDNA-originated and integrated genome-derived transcripts, the results showed that the relative amount of transcripts from cccDNA relative to the integrated HBV genome was 10-20%. Thus, surprisingly, the transcriptional ability of cccDNA is dramatically less efficient than that of the integrated genome in our established cell lines. The degree of inefficiency seemed to range from 10% compared to the integrated genome (4 cccDNA copies, 2 integrated copies) to as low as 2% (6 cccDNA copies, 1 integrated copy). It is known that cccDNA forms a compact minichromosome-like structure within the hepatocyte (Bock et al., 2001, J. Mo.l Biol. 307:183-96.). This inactive form may partially explain why the transcription rate of the cccDNA is so ineffective. There is a need elucidate the regulatory factors which participate in cccDNA-associated transcription.

### EXAMPLE 3

To date, several transcriptional factor binding elements have been located and investigated within the viral genome/cccDNA. These transcriptional factors, including AP-1, AP-2, NF-kB, C/EBP, ATP/CREB, SRF and SP1, could interact with the viral genome/cccDNA and be involved in the regulation of HBV gene transcription (Caselmann et al., 1995, J. Hepatol. 22:34-7; Doria et al., 1995, EMBO J 14:4747-57; Henkler et al., 1996, J. Viral Hepat. 3:109-21; and Kekule et al., 1993, Nature 361:742-5). As these cellular transcriptional may activate HBx, the above-described assay was used to examine regulation of cccDNA by HBx.

To eliminate expression of HBx from p1.3HBcl, a AUG mutation and a stop codon were introduced to p1.3HBcl to form 1.3HBclI-X⁻. This plasmid was co-transfected with plasmids encoding wild type HBX (pX) or mutant HBX p(HBx (X-Δ(100-115)) into HepG2 cells in the manner described above. The pHBx (X-Δ(100-115)) plasmid encoded a mutant HBx containing internal deletion of amino acids 100-115, which were essential for the transactivation ability (Arii et al., 1992, Oncogene 7:397-403 and Takada et al., 1994, Virology 205:503-10).

The above-described RT-PCR/BclI digestion assay was used to monitor the BclI-containing PCR products. In the cells transfected with 1.3HBclI-X⁻ only, no cccDNA transcription was found. This was trans-complemented by co-transfection of pX but not by pHBx (X-□(100-115)). The result suggested that trans-activation function of HBx is required for production of cccDNA-derived RNA.

Two scenarios could explain why the transcripts from the cccDNA were eliminated in absence of transactivation domain of HBx. First, the functionally inactive truncated HBx protein might fail to support cccDNA formation. Second, the transactivation function of the HBx protein might be responsible for the transcriptional regulation of the cccDNA. To exclude the first possibility, several HBx null mutants were obtained by stably transfecting cells with an HBx-mutated 1.3-fold HBV genome. Southern blot analysis of cccDNA from one HBx-null line, 1.3.2×m5, revealed a DNA molecule that migrated as cccDNA. Similar result of cccDNA production was also observed in another HBx-null clone, 1.3.2×m1. These HBx-null lines are able to produce cccDNA molecules in the same way as Hep2.2.15, a well established HBV-producing cell line (Sells et al., 1987, Proc. Natl. Acad. Sci. USA 84:1005-9). This RT-PCR/BclI digestion result for these HBx-null lines further confirmed the observation described above that the transcripts from cccDNA were down-regulated in the absence of HBx. Taking together, the results suggested that HBx is essential for optimal transcription activity of the cccDNA. HBx may enhance the transcriptional ability of cccDNA by elevating cellular transcription factors expression. These results for the first time provide evidence that HBx is involved in the transcription regulation of the cccDNA.

It has been reported that woodchuck hepatitis virus (WHV) genome harboring the X mutation could not establish an infection cycle after the viral genome was delivered into the woodchuck liver (Chen et al., 1993, J. Virol. 67:1218-26 and Zoulim et al., 1994, J. Virol. 68:2026-30). On the basis of the current HBV life cycle, the cccDNA molecule is the most pivotal of the DNA intermediates in the establishment of the infection cycle in vivo. The above results of HBx suggest that the inability to establish an infection cycle in X-mutated WHV may due to inefficient transcription from the cccDNA preceding virus production. Since the expression of viral pregenomic RNA is down-regulated in the absence of X protein, the re-entry of the viral genome into nucleus and the amplification of the cccDNA pool may then be eliminated indirectly. In this scenario, the viral transactive protein, HBx, may therefore play a critical role in the accumulation of cccDNA in a natural infection and thus also in the establishment of viral infection.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims

What is claimed is:

1. An isolated nucleic acid comprising a mutant hepatitis B virus genome, wherein the mutant hepatitis B virus genome contains a heterologous DNA sequence in the 5′ terminus redundancy region, the heterologous DNA sequence being 1 to 50 nucleotides in length.

2. The nucleic acid of claim 1, wherein the heterologous DNA sequence is 1 to 20 nucleotides in length.

3. The nucleic acid of claim 1, wherein the heterologous DNA sequence is 1 to 10 nucleotides in length.

4. The nucleic acid of claim 1, wherein the mutant hepatitis B virus genome is HBx deficient.

5. The nucleic acid of claim 1, wherein the heterologous DNA sequence contains a restriction enzyme site.

6. The nucleic acid of claim 5, wherein the mutant hepatitis B virus genome is HBx deficient.

7. The nuclei acid of claim 6, wherein the restriction enzyme site is a BclI site.

8. The nucleic acid of claim 5, wherein the heterologous DNA sequence is 1 to 20 nucleotides in length.

9. The nucleic acid of claim 8, wherein the heterologous DNA sequence is 1 to 10 nucleotides in length.

10. The nucleic acid of claim 9, wherein the heterologous DNA sequence contains a restriction enzyme site.

11. A vector comprising a nucleic acid of claim 1.

12. A host cell comprising a nucleic acid of claim 1.

13. The host cell of claim 12, wherein the cell is a HepG2 cell or a HuH-7 cell.

14. A method of monitoring transcription of a hepatitis B virus covalently closed circular DNA, comprising
obtaining a host cell containing a nucleic acid of claim 1;
culturing the host cell to allow for transcription of a hepatitis B virus covalently closed circular DNA to generate a population of RNA transcripts, each containing in the 3′ terminus redundancy region an heterologous RNA sequence that is complementary to the heterologous DNA sequence or the complement thereof; and
determining a level of the transcripts.

15. The method of claim 14, wherein the level of the transcripts is determined by RT-PCR.

16. The method of claim 15, wherein the RT-PCR generates a DNA product that spans the heterologous RNA sequence.

17. The method of claim 16, wherein the heterologous DNA sequence contains a BclI site.

18. The method of claim 17, wherein the DNA product is subjected to digestion of BclI.

19. The method of claim 18, wherein the DNA product is further subjected to digestion of SspI.

20. The method of claim 14, wherein the host cell is a HepG2 cell or a HuH-7 cell.

21. The method of claim 14, wherein each of the transcripts contains a second heterologous RNA sequence in the 5′ terminus redundancy region, the second heterologous RNA sequence being identical to the heterologous RNA sequence in the 3′ terminus redundancy region.

22. A screening method of identifying a test compound for treating a chronic infection with hepatitis B virus, the method comprising:
obtaining a host cell containing a nucleic acid of claim 1;
contacting the host cell with a test compound in a medium;
culturing the host cell to allow for transcription of a hepatitis B virus covalently closed circular DNA to generate a population of RNA transcripts, each containing in the 3′ terminus redundancy region a heterologous RNA sequence that is complementary to the heterologous DNA sequence or the complement thereof; and
determining a level of the transcripts, wherein the compound is determined to be effective in treating the infection if the level of the transcript is lower than that determined in the same manner from a second cell except that the second cell is incubated in a medium free of the compound.

23. The method of claim 22, wherein the compound is a small molecule compound, a nucleic acid, or a peptide.

24. The method of claim 23, wherein the peptide is an oligopeptide or a polypeptide.

25. The method of claim 22, wherein the level of the transcripts is determined by RT-PCR.

26. The method of claim 25, wherein the RT-PCR generates a DNA product that spans the heterologous RNA sequence.

27. The method of claim 26, wherein the heterologous DNA sequence contains a BclI site.

28. The method of claim 27, wherein the DNA product is subjected to digestion of BclI.

29. The method of claim 28, wherein the DNA product is further subjected to digestion of SspI.

30. The method of claim 22, wherein the host cell is a HepG2 cell or a HuH-7 cell.

31. The method of claim 22, wherein each of the transcripts contains a second heterologous RNA sequence in the 5′ terminus redundancy region, the second heterologous RNA sequence being identical to the heterologous RNA sequence in the 3′ terminus redundancy region.
